(19) United States
(12) Patent Application Publication
(10) Pub. No.: US 2022/0331144 A1
(43) Pub. Date: Oct. 20, 2022

(54) Undergarment for Colostomy Patients

(71) Applicant: Nicole Richards, Zimmerman, MN (US)

(72) Inventor: Nicole Richards, Zimmerman, MN (US)

(21) Appl. No.: 17/234,765

(22) Filed: Apr. 19, 2021

(51) Int. Cl. A61F 5/44; A61F 5/449; A41B 9/00; A41B 9/14

(52) U.S. Cl. CPC A61F 5/4408; A61F 5/449; A41B 9/007; A41B 9/14; A41B 2400/32

(57) ABSTRACT

The present invention provides a two-piece undergarment system with a pocket to hold the colostomy bag and allowing easy wearing and removal of a part of the garment.

### FIELD OF THE INVENTION

The present invention relates to a garment to be worn by people for supporting an ostomy/colostomy bag attached to an opening called stoma on their abdominal wall following a surgery. The invention, in particular relates to an undergarment with a pocket to hold the ostomy bag and allowing easy wearing and removal of a part of the garment.

### BACKGROUND OF THE INVENTION

Colostomy is a surgical procedure which leaves an opening called stoma in the abdominal wall of the patients. Patients who undergo such a procedure need to wear a colostomy bag attached to the stoma wherein all the bodily waste is discharged. The colostomy bag is either permanent or temporary for some. It changes the lives of people who undergo such surgeries. There is always a need for garments to normalize the lives of patients of colostomy as much as possible. However, all the garments or solutions that exist make it difficult for the patients to even perform normal roles like relieving oneself in the toilet.

Further, one needs easy and fast access to the colostomy bag while on the go for emptying it for the waste that gets stored in there. Usually, it is a cumbersome process to even empty and clean the bag when outside.

The present invention provides a two-piece undergarment system wherein the segment that covers the pelvic area can be removed to relieve oneself without exposing the colostomy bag and the other segment holds and supports the colostomy bag.

### SUMMARY OF THE INVENTION

This Summary is provided to introduce a selection of concepts in a simplified form that are further described below in the Detailed Description of the invention. This Summary is not intended to identify key aspects or essential aspects of the claimed subject matter. Moreover, this Summary is not intended for use as an aid in determining the scope of the claimed subject matter.

The present application discloses an undergarment that supports and hides the colostomy bag attached to the stoma thereby providing discreteness for the patients.

It is an object of this invention to provide a two-piece undergarment system that not only hides the colostomy bag, but also allows easy access to the pelvic area without exposing the colostomy bag.

It is an additional object of the invention to provide a two-piece undergarment system comprising an upper segment and a lower segment of the undergarment, wherein the upper segment of the undergarment acts as a protective belt or waistband around the waist of the user which holds and supports the colostomy bag, whereas the lower segment of the undergarment can be detached from the upper segment of the undergarment, thereby providing easy access to both the colostomy bag and genitals of the patient without removing the entire undergarment.

The upper segment and lower segment of the undergarment are made up of a fabric or combination of fabrics wherein the fabric forming the protective waistband is preferably thick and elastic in order to provide pressure and good support for holding the colostomy bag whereas the fabric in the lower segment is soft, stretchable, flexible and covers the lower pelvic area of the user.

It is another object of this invention to provide an undergarment made up of a fabric that consists of bamboo, cotton, polyester or spandex.

It is a further object of this invention to provide a two-piece undergarment system comprising a pocket on the outside of the fabric of the protective belt or waistband to hold the colostomy bag.

It is still another object of this invention to provide a protective belt or waistband with a pocket consisting of a pouch formed by two layers of fabric of the belt to hold the colostomy bag within it. The feature of the pouch on the waistband which holds the colostomy bag provides a layer of fabric between the skin of the user and the colostomy bag thereby preventing any skin irritation or inflammation which may happen with prolonged use of a colostomy bag attached to a hole or stoma on your body. Furthermore, the pouch holds the bag in its place all the time even when it gets detached from the hole or stoma on the body thereby preventing any embarrassing situations for the user.

The pocket on the protective belt further consists of a vertical opening or slit sewn on the outside fabric of the belt for insertion of the colostomy bag into the pouch, a flap cover on the outside fabric to cover the vertical opening or slit and a snap enclosure on the flap cover to close the flap cover onto the fabric. The features of flap cover and snap enclosure provide extra support to keep the colostomy bag in its place in the pouch and the snap enclosure prevents the bag from coming or falling out of the pouch. Furthermore, a tube hole, in the pouch and on the inside of the protective belt, is provided to be aligned with the stoma or insertion on the body of the patient.

Both upper and lower segments of the undergarment are provided with plurality of snap fasteners in front and back of each segment to attach or detach the lower segment of the undergarment from the upper segment of the undergarment.

Further, the plurality of snap fasteners on each segment of the undergarment consists of male snap fasteners affixed at the bottom of the fabric of the protective waistband and female snap fasteners affixed at the top of the fabric forming the lower segment of the undergarment. The snap fasteners make the fastening and unfastening user friendly wherein the user can easily detach or attach the segments easily and at will anytime, anywhere they desire. In one embodiment, the user can drop down the front lower segment by unfastening the female snaps on the lower segment from the male snaps on the upper segment, while keeping the back lower segment attached, to provide more accessibility to the crotch area of the user without taking off the entire undergarment.

It is yet another object of this invention to provide a level of discreteness for females who have undergone a colostomy procedure.

In a preferred embodiment, a two-piece undergarment system comprises an upper segment as a protective waistband to hold and support the colostomy bag, wherein the protective waistband consists of a thick layer of fabric around the waist of the user, and a pocket located on the exterior of the protective waistband to house the colostomy bag; a lower segment, wherein the lower segment of the garment consists of a layer of fabric covering the pelvic area of the user; at least a few snap fasteners at the front and back of the upper segment and lower segment to attach and detach the lower segment from the upper segment wherein the upper segment consists of male snaps at the bottom of the protective waistband and the lower segment consists of female snaps at the top of the fabric.

These together with other objects and advantages which will become subsequently apparent reside in the details of the invention as more fully described and claimed, reference being had to the accompanying drawings forming a part thereof, wherein like numerals refer to like parts throughout.

### BRIEF DESCRIPTION OF THE DRAWINGS

The novel features of the invention, as well as the invention itself, both as to its structure and its operation will be best understood from the accompanying drawings, taken in conjunction with the accompanying description, in which similar reference characters refer to similar parts, and in which:

FIG. 1: shows a front view of a preferred embodiment of an undergarment according to the invention;

FIG. 2: shows a right side view of a preferred embodiment of an undergarment according to the invention;

FIG. 3: shows a front view of detached upper and lower segments of an undergarment according to the invention;

FIG. 4: shows a half detached lower segment of an undergarment according to the invention.

### DETAILED DESCRIPTION OF THE INVENTION

What follows is a description of various aspects, embodiments and/or examples in which the invention may be practiced. Reference will be made to the attached drawings, and the information included in the drawings is part of this detailed description. The aspects, embodiments and/or examples described herein are presented for exemplification purposes, and not for limitation purposes. It should be understood that structural and/or logical modifications could be made by someone of ordinary skill in the art without departing from the scope of the invention.

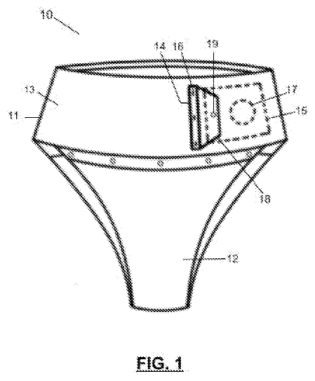

FIG. 1 shows a full front view of an undergarment according to the present invention. As shown in FIG. 1, a two-piece undergarment (10) of the present invention comprises an upper segment (11) and a lower segment (12), a protective waistband (13), a pocket (14), a pouch (15), a vertical opening or slit (16), a tube hole (17), a flap cover (18), and a snap enclosure (19).

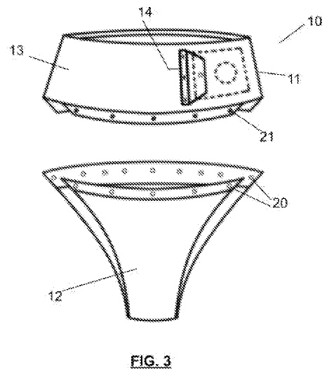

As illustrated in FIGS. 1, 3 & 4, the upper segment (11) of the undergarment (10) made up of a thick layer of fabric forms a protective waistband (13) which extends around the waist of the user. The said waistband (13) includes a pocket (14) which holds and supports the colostomy bag in its place. The protective waistband (13) formed in the upper segment (11) of the garment (10) is preferably constructed with two layers of fabric to form a cavity or pouch (15) within a pocket (14) to house the colostomy bag inserted through the vertical opening (16) formed on the exterior of the waistband (13). In one embodiment, the whole protective waistband is made up of two layers of fabric. In another embodiment, only the pouch on the waistband is made up of two layers of fabric in order to create a cavity to hold the colostomy bag.

The pocket (14) further includes a flap cover (18) on the exterior of the waistband and the said flap cover (16) covers the vertical opening (16) by means which may include, but not limited to, a snap or Velcro to form a snap enclosure (19) as shown in FIG. 1. The snap enclosure helps in keeping the bag in the pouch and prevents it from falling or coming out even when it gets detached from the stoma on the abdomen of the user. As illustrated in FIGS. 1, 3 & 4, the pocket (14) also includes a tube hole (17) on one side of the pouch (15) to be aligned with the stoma or hole on the body of the user.

In one embodiment, the said pocket on the exterior of the fabric of the upper segment of the undergarment can be on the right or left side of the garment depending upon the location of the stoma.

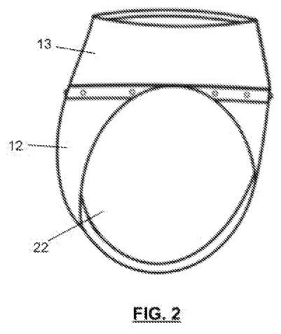

According to the present invention, the lower segment (12) of the undergarment (10) can be detached from the upper segment (11) of the undergarment (10) whenever the user desires an access to her genitals. The lower segment (12) of the undergarment (10) is made up of a sheet of fabric that extends from bottom of the upper segment in front to the bottom of the upper segment in rear thereby forming leg opening (22), as shown in FIG. 2, on each side and covering the entire pelvic area of the user.

Further, the top strap of lower segment (12) and bottom strap of the upper segment (11) at the back and front are provided with snap fasteners for easy attachment and detachment of both the segments from each other.

The snap fasteners on upper and lower segments respectively hold the lower segment in its place and also helps in attaching and detaching the lower segment from the upper segment of the undergarment. The snap fasteners located on the upper and lower segment of the undergarment include but not limited to plastic or metal snap buttons, tape, hooks, Velcro. In one embodiment, as illustrated in FIG. 3, the lower segment (12) is provided with, 1 to 5 male snaps (20) in the front and 1 to 8 male snaps, in the back of the upper segment of the undergarment, whereas 1 to 5 female snaps (20) in the front and 1 to 8 female snaps (20) in the back of the lower segment (12) of the undergarment (10) are used to attach and detach the lower segment from the upper segment. In another embodiment, the female snaps are provided on the upper segment and males snaps are provided on the lower segment of the undergarment.

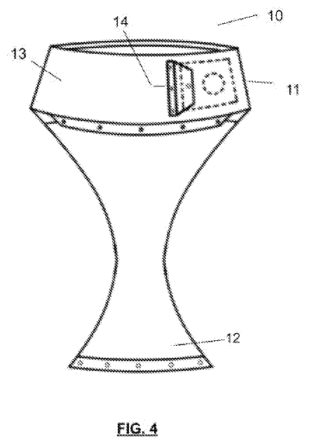

As illustrated in FIGS. 3 & 4, the lower segment (12) of the undergarment (10) of the present invention can be detached and still have the waistband or belt (13) on the body to retain or cover the colostomy bag. In one embodiment, the lower segment (12) of the undergarment (10) of the present invention detaches completely from the upper segment (11) of the undergarment (10), as shown in FIG. 3. The removal of only the lower segment of the undergarment helps the user maintain a normal lifestyle, for e.g. the lower segment can be removed during any medical examination of the pelvic area or during intimate moments without disturbing or worrying about the colostomy bag. In another embodiment, as shown in FIG. 4, the said lower segment (12) can be detached halfway in front, wherein, the back portion of the lower segment (12) remains hooked or snapped at the back with the upper segment (11) of the undergarment (10). The halfway drop down of the lower segment of the undergarment allows the user to relieve oneself without removing the whole undergarment.

The upper segment and lower segment of the undergarment are made up of a fabric or combination of fabrics. According to the present invention, the fabric of the upper and lower segment preferably consists of bamboo, cotton, polyester or spandex. Bamboo provides many benefits for patients who have undergone a colostomy procedure and the benefits include anti-microbial properties, and good wicking properties for the wound whereas spandex material provides elasticity and stretchability to an undergarment.

The undergarment of the present invention can be designed for both male and female users and moreover, the said undergarment works for all kinds of drainable and closed colostomy bags. Further, the pouch that houses the colostomy bag can fit bags with variable lengths, shapes and sizes. Furthermore, the undergarment of the present invention can be manufactured for daily wear or can be designed with laces and/or sheer for fancy wear for female users. In addition, the undergarment of the present invention can be made in different varieties including, but not limited to, classic briefs, high-cut briefs, control briefs, hipsters, bikinis, and the like.

The feature of the protective waistband with the pocket and pouch therein, which helps not only in hiding the bag but also supporting and keeping the colostomy bag in its place, and the detachment of the lower segment of the undergarment from the upper segment of the undergarment without removing the whole undergarment, helps in normalizing intimate moments for the patients.

While many of the fundamental characteristics and features of the undergarment have been described herein, with reference to particular embodiments thereof, a latitude of modification, various changes and substitutions are intended in the foregoing disclosure, and it should be apparent that in some instances some features of the invention will be employed without a corresponding use of other features without departing from the scope of the invention as set forth. It should be understood that such substitutions, modifications, and variations may be made by those skilled in the art without departing from the spirit and scope of the invention. Consequently, all such modifications and variations are included within the scope of the invention as defined herein.

What is claimed is:

1. A two-piece undergarment system comprising:
an upper segment as a protective waistband to hold and support the colostomy bag, wherein the protective waistband consists of a thick layer of fabric around the waist of the user, and a pocket located on the exterior of the protective waistband to house the colostomy bag;
a lower segment, wherein the lower segment of the garment consists of a layer of fabric covering the pelvic area of the user;
plurality of snap fasteners at the back and front of both the upper segment and the lower segment to attach and detach the lower segment from the upper segment, wherein the upper segment consists of male snaps affixed at the bottom of the protective waistband and the lower segment consists of female snaps affixed at the top of the fabric.

2. The two-piece undergarment of claim 1, wherein the pocket located on the outside fabric of the protective waistband to house the colostomy bag further consists of a pouch formed by two layers of fabric to hold the colostomy bag, a tube hole sewn on the layer of the fabric closest to the skin of the user and aligned with the stoma or insertion on the body of the user, a vertical opening sewn on the outside fabric of the waistband for the insertion of colostomy bag into the pouch, a flap cover to cover the vertical opening, and a snap enclosure on the flap cover to close the flap cover onto the fabric thereby providing extra support to keep the colostomy bag in its place in the pouch.

3. The two-piece undergarment of claim 1, wherein the pocket on the exterior of the protective waistband can be on the left or right side of the upper segment.

4. The two-piece undergarment of claim 1, wherein the lower segment attaches and detaches completely from the upper segment as desired by the user without exposing the colostomy bag.

5. The two-piece undergarment of claim 1, wherein the lower segment detaches halfway in front exposing the crotch area of the user without exposing the colostomy bag.

6. The two-piece undergarment of claim 1, wherein the fabric of the upper segment of the undergarment is preferably in two layers to provide thickness for pressure and support for holding the colostomy bag.

7. The two-piece undergarment of claim 1, wherein the fabric of the lower segment of the undergarment is soft and stretchable fabric to cover the entire pelvic area of the user.

8. The two-piece undergarment of claim 1, wherein the fabric of the upper and lower segment preferably consists of bamboo, cotton, polyester, or spandex.

9. The two-piece undergarment of claim 1, wherein the number of male snaps on the bottom strap of the upper segment in the front are preferably 1 to 5.

10. The two-piece undergarment of claim 1, wherein the number of male snaps on the bottom strap of the upper segment in the back are preferably 1 to 8

11. The two-piece undergarment of claim 1, wherein the number of female snaps on the top strap of the lower segment in the front are preferably 1 to 5.

12. The two-piece undergarment of claim 1, wherein the number of female snaps on the top strap of the lower segment in the back are preferably 1 to 8.
